(19) United States
(12) Patent Application Publication
(10) Pub. No.: US 2006/0058821 A1
(43) Pub. Date: Mar. 16, 2006

(54) Tongue scraper

(76) Inventor: John M. Jansheski, Maryville, TN (US)

Correspondence Address: LUEDEKA, NEELY & GRAHAM, P.C., P O BOX 1871, KNOXVILLE, TN, 37901, US

(21) Appl. No.: 10/941,762

(22) Filed: Sep. 15, 2004

(51) Int. Cl. A61B 17/24

(52) U.S. Cl. 606/161

(57) ABSTRACT

A device for cleaning the tongue, including an elongate handle and a head portion extending away from the handle, the head portion including a pair of legs, a connector extending between terminal ends of the legs, and a substantially continuous layer of a thermoplastic elastomeric material overlying the legs and the connector to define a tongue contact surface, with the portions of the tongue contact surface overlying the legs having different flexibility than the portion of the tongue contact surface overlying the connector.

### FIELD OF THE INVENTION

This invention relates generally to oral hygiene devices. More particularly, the invention relates to devices for scraping the tongue for cleaning thereof.

### BACKGROUND AND SUMMARY OF THE INVENTION

It has been observed that bad breath may result from volatile sulfur compounds. These compounds are typically produced by bacteria in the mouth. The back of the tongue is typically an area of the mouth that is difficult to clean by normal brushing techniques. Accordingly, such compounds often remain on the back of the tongue and result in bad breath.

The present invention relates to a device for cleaning the tongue, as by scraping the tongue in a manner designed to remove volatile sulfur compounds from the back of the tongue.

In a preferred embodiment, the scraper includes an elongate handle and a head portion extending away from the handle. The head portion includes a pair of legs, a connector extending between terminal ends of the legs, and a substantially continuous layer of a thermoplastic elastomeric material overlying the legs and the connector to define a tongue contact surface. The portions of the tongue contact surface overlying the legs have different flexibility characteristics than the portion of the tongue contact surface overlying the connector.

Preferably, the portion of the tongue contact surface overlying the connector is substantially more flexible than the other portions.

### BRIEF DESCRIPTION OF THE DRAWINGS

Further features of preferred embodiments of the invention will become apparent by reference to the detailed description of preferred embodiments when considered in conjunction with the figures, wherein like reference numbers, indicate like elements through the several views, and wherein,

FIG. 1 is a perspective view of a tongue scraper according to a preferred embodiment of the invention.

FIG. 2 is a top plan view of the scraper of FIG. 1.

FIG. 3 is a bottom plan view of the scraper of FIG. 1.

FIG. 4 is a right side view of the scraper of FIG. 1, the left side being identical thereto.

FIGS. 5 and 6 are opposite end views of the scraper of FIG. 1.

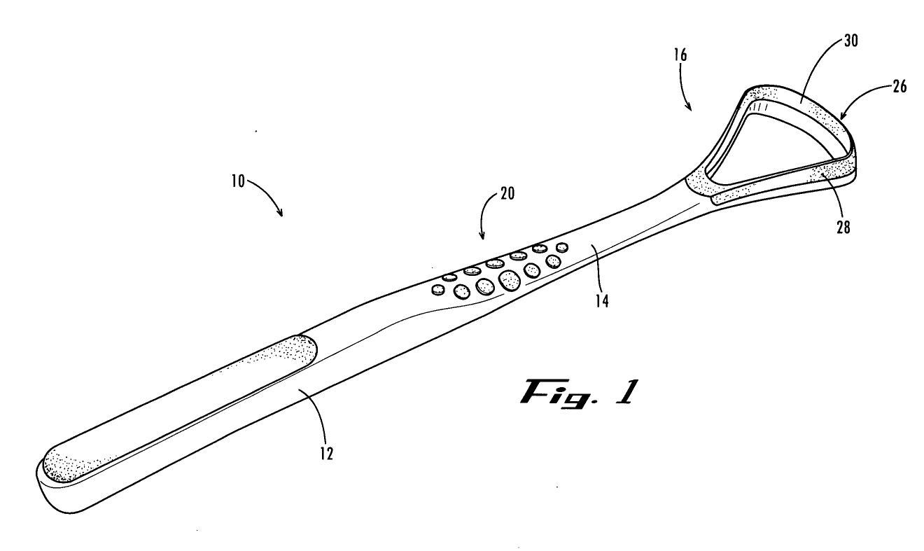

### DETAILED DESCRIPTION

With reference to the drawings, the invention relates to a tongue scraper 10 having a handle 12, a neck 14, and a head 16 extending from the neck 14. The scraper 10 is desirably configured to facilitate removal of volatile sulfur compounds from the back of the tongue.

The handle 12 is preferably made as by molding of a polymeric material such as nylon, plastic, or the like and is configured for grasping by the hand of a user and preferably includes grip enhancing features such as a grip surface having raised ribs 18 or projections 20 preferably made of a soft, flexible material such as a thermoplastic elastomer, most preferably styrene-ethylene-butylene-styrene (SEBS). The grip enhancing features are preferably co-molded with the handle, with the grip enhancing features preferably having configurations and contrasting coloration to provide an aesthetically pleasing appearance. For example, the handle is preferably a light coloration, such as white, and the grip enhancing features a re preferably a darker color, such as blue.

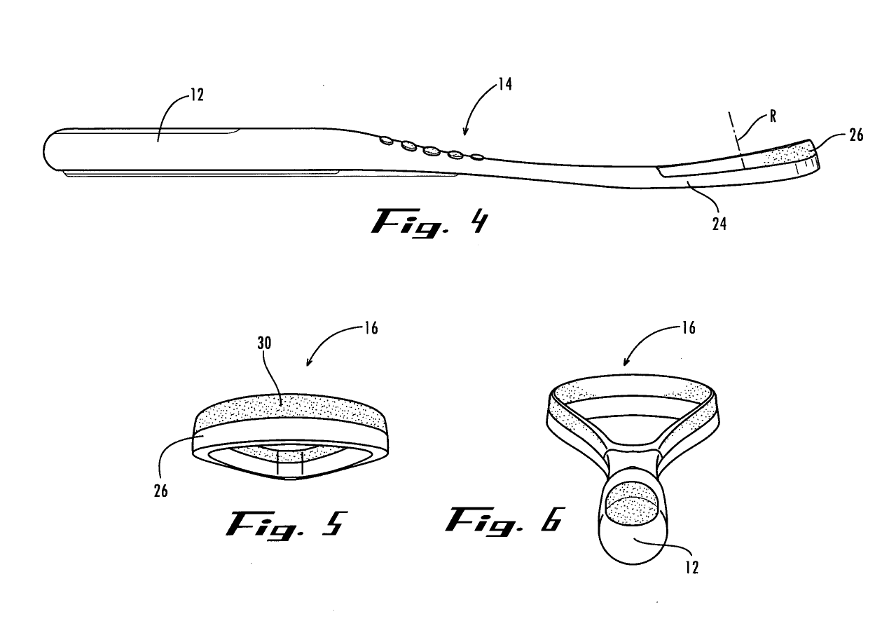

The neck 14 extends from the handle 12 and has a low or slim profile as best seen in FIG. 4 that advantageously enables the cleaner 10 to access back portions of the tongue of a user and help avoid triggering of a gag reflex. The dimensions of the neck 14 also render the neck substantially flexible so that the neck flexes slightly when the scraper 10 is urged against the tongue during use. In this regard, the neck 14 preferably has a thickness of about ⅛ inch.

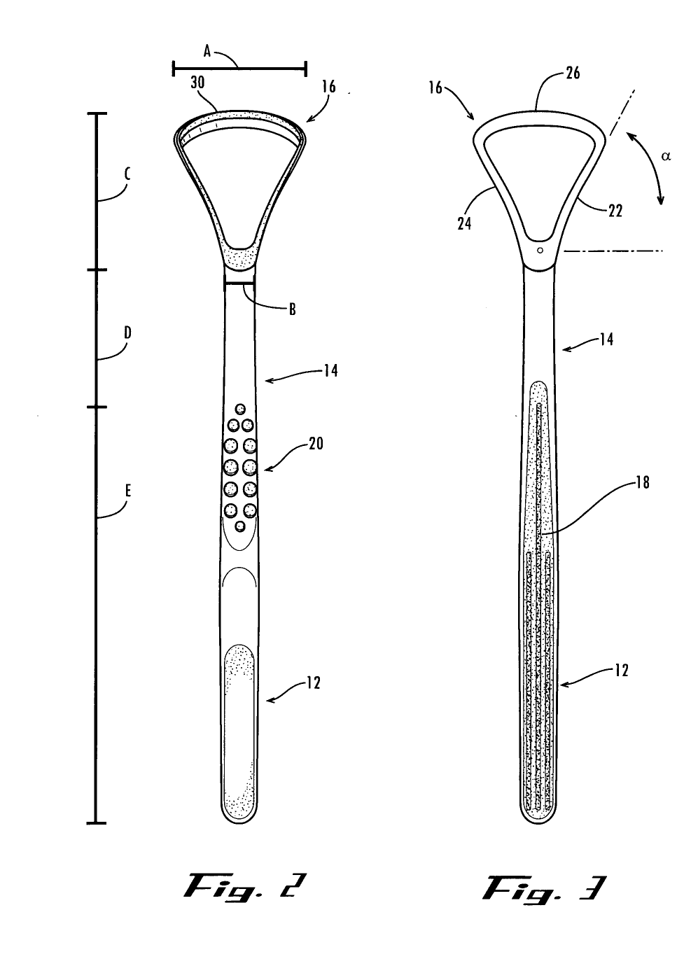

The head 16 is desirably configured to be relatively narrow in width so as to facilitate insertion thereof into the mouth of a user. In this regard, the head 16 is substantially pie-shaped and includes a pair of substantially linear legs 22 and 24. The terminal ends of the legs 22 and 24 are joined by an arc-shaped connector 26 which provides a rounded terminal end of the head 16. With reference to FIGS. 2 and 3, the legs 22 and 24 extend angularly outwardly from the neck 14 at an angle a of preferably about 30 degrees. As seen in FIG. 4, the legs 22 and 24 also preferably curve upwardly away from the longitudinal plane of the neck 14, with a radius of curvature R.

The head 16 also preferably includes, as its uppermost or tongue-contacting surface, a substantially continuous layer 28 of a thermoplastic elastomeric material, such as SEBS. The layer 28 is preferably of substantially uniform height or vertical thickness so as to provide a substantially uniformly smooth upper surface. However, the width or lateral thickness of the portions of the layer 28 is preferably substantially greater adjacent the neck 14 and along the lengths of the legs 22 and 24, as compared the width of the portion of the layer 28 overlying the arc 26, which preferably tapers as it rises from the arc 26 to define a cleaning edge 30.

The portions of the layer 28 adjacent the neck 14 and along the lengths of the legs 22 and 24 are preferably sufficiently uniformly thick so as to not substantially flex when pressed against the tongue during use. Conversely, the portion of the layer 28 overlying the arc 26 and the cleaning edge 30 defined thereby is preferably sufficiently thin so as to substantially flex when pressed against the tongue during use.

The configuration of the layer 28 in this manner provides a substantially soft contact surface for contacting the tongue having both flexible and rigid contact characteristics. Thus, if more flexible cleaning characteristics are desired, the tongue is preferably contacted with the cleaning edge 30. However, if more aggressive cleaning contact is desired, the portions of the surface 28 overlying the legs 22 and 24 may be utilized.

It will be understood that the scraper 10 may be provided in a variety of dimensions, with the preferred dimensions being selected to correspond to the size of the mouth with which the scraper is to be used. However, for the purpose of example only, and with reference to FIG. 2, the scraper 10 preferably has the following dimensions:

| Dimension | Distance (inch) |
|---|---|
| A | 1 3/16 |
| B | ¼ |
| C | 1½ |
| D | 1½ |
| E | 4 |

The foregoing description of certain exemplary embodiments of the present invention has been provided for purposes of illustration only, and it is understood that numerous modifications or alterations may be made in and to the illustrated embodiments without departing from the spirit and scope of the invention as defined in the following claims.

What is claimed is:

1. A device for cleaning the tongue, comprising an elongate handle and a head portion extending away from the handle, the head portion including a pair of legs, a connector extending between terminal ends of the legs, and a substantially continuous layer of a thermoplastic elastomeric material overlying the legs and the connector to define a tongue contact surface, with the portions of the tongue contact surface overlying the legs having different flexibility than the portion of the tongue contact surface overlying the connector.

2. The device of claim 1, wherein the flexibility of the portions of the tongue contact surface overlying the legs is substantially less than the flexibility of the portion of the tongue contact surface overlying the connector.

3. The device of claim 1, wherein the portion of the tongue contact surface overlying the connector is tapered.

4. The device of claim 1, wherein the head is substantially pie-shaped, with the connector being an arc.

5. The device of claim 1, further comprising a flexible neck intermediate the head and the handle.

6. The device of claim 1, wherein the thermoplastic elastomeric material comprises styrene-ethylene-butylene-styrene.
